(19) United States
(12) Patent Application Publication
(10) Pub. No.: US 2026/0217658 A1
(43) Pub. Date: Jul. 30, 2026

(54) ONE STEP REACTION FOR THE CATALYTIC SYNTHESIS OF CYCLIC UREA DERIVATIVES

(71) Applicant: HUNTSMAN PETROCHEMICAL LLC, The Woodlands, TX (US)

(72) Inventor: Hui ZHOU, The Woodlands, TX (US)

(21) Appl. No.: 19/146,550

(22) PCT Filed: Jan. 9, 2024
(86) PCT No.: PCT/US2024/010782

§ 371 Date: Jul. 9, 2025

Related U.S. Application Data
(60) Provisional application No. 63/439,899, filed on Jan. 19, 2023.

(51) Int. Cl. C07D 233/32

(52) U.S. Cl. CPC C07D 233/32

(57) ABSTRACT

A process for preparing a cyclic urea derivative by reacting an alkylene carbonate and a primary amine in the presence of a metal oxide catalyst to form the cyclic urea derivative.

### CROSS-REFERENCE TO RELATED APPLICATION

This application claims priority to U.S. Provisional No. 63/439,899 filed Jan. 19, 2023. The noted applications are incorporated herein by reference.

### FIELD

The present disclosure generally relates to a process for preparing cyclic urea derivatives from an alkylene carbonate and a primary amine in the presence of a metal oxide catalyst and their subsequent use in various applications, such as in lithium ion battery fabrication, the removal of paint and coatings, and as a solvent in compositions useful in the electronics, automotive, agricultural and pharmaceutical industries.

### BACKGROUND

N-methyl-2-pyrrolidone (NMP) has been widely used in various industries, especially in the semiconductor, battery, and other electronics industries. NMP is a polar organic chemical with a unique combination of properties, such as low vapor pressure, comparatively high flash point, low freezing point and high boiling point. The molecular structure of NMP provides a particular combination of dispersive, polar, and hydrogen bonding forces that enable its unique solvency. However, the European Chemicals Agency (ECHA) has classified NMP as a “Substance of Very High Concern” because of its reproduction toxicity. Accordingly, there is a pressing need to replace NMP with lower toxicity alternatives having similar performance characteristics.

One potential alternative includes cyclic urea derivatives. Various processes are known for producing such derivatives, such as by: reacting carbon dioxide in the presence of (i) an oxide of an element of main group three or four or subgroup two to six of the Periodic Table, (ii) a mixture of these, or (iii) an aluminum silicate or magnesium silicate (see U.S. Pat. No. 4,897,480); reacting ethylene carbonate with a primary amine without the necessity of using solvents or catalysts (see U.S. Pat. No. 5,783,706); and reacting an alkylene oxide with at least one of (i) carbon dioxide and a monoalkylamine, (ii) a carbon dioxide compound of a monoalkylamine and (iii) a 1,3-dialkylurea at a temperature of 50° C. or higher. However, problems associated with known processes include for instance, harsh processing conditions, long processing times, production of unwanted by-products, and low yields of the cyclic urea derivative.

Therefore, there is a need for the development of a relatively simple, efficient process for the preparation of cyclic urea derivatives that does not suffer from the disadvantages of state of the art processes.

### SUMMARY

The present disclosure generally provides a process for preparing a cyclic urea derivative having the general formula

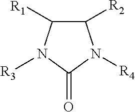

where R₁ and R₂ are independently selected from (a) hydrogen, (b) a straight-chain, branched or cyclic alkyl radical having 1 to 12 carbon atoms, which can be optionally substituted by C₆-C₁₀ aryl, F, Cl or Br, (c) a C₆-C₁₀ aryl radical, which can be optionally substituted by C₁-C₁₂ alkyl, (d) a heteroalkyl radical in which the alkyl radicals as defined in (b) are interrupted by one or more heteroatoms selected from O, S and N, and (e) a heteroaryl radical with 5 to 10 ring atoms which contain 1 to 3 heteroatoms selected from O, S and N and R₃ and R₄ are independently selected from (a) a straight-chain, branched or cyclic alkyl radical having 1 to 12 carbon atoms, which can be optionally substituted by C₆-C₁₀ aryl, F, Cl or Br, (b) a C₆-C₁₀ aryl radical, which can be optionally substituted by C₁-C₁₂ alkyl, (c) a heteroalkyl radical in which the alkyl radicals as defined in (a) are interrupted by one or more heteroatoms selected from O, S and N, and (d) a heteroaryl radical with 5 to 10 ring atoms which contain 1 to 3 heteroatoms selected from O, S and N. The process generally includes the step of reacting an alkylene carbonate and a primary amine in the presence of a metal oxide catalyst to form a reaction product comprising the cyclic urea derivative.

The cyclic urea derivatives produced according to the process of the present disclosure may be used in various applications, such as in the fabrication of lithium ion batteries, to remove paint and coatings, and as a solvent for compositions useful in the electronics, automotive, agricultural and pharmaceutical industries.

### DETAILED DESCRIPTION

Before explaining aspects of the present disclosure in detail, it is to be understood that the present disclosure is not limited in its application to the details of construction and the arrangement of components or steps or methodologies set forth in the following description. The present disclosure is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the present disclosure shall have the meanings that are commonly understood by those having ordinary skill in the art. Further, unless otherwise required by context, singular terms shall include pluralities and plural terms shall include the singular.

As utilized in accordance with the present disclosure, the following terms, unless otherwise indicated, shall be understood to have the following meanings.

The use of the word “a” or “an”, when used in conjunction with the term “comprising”, “including”, “having”, or “containing” (or variations of such terms) may mean “one”, but it is also consistent with the meaning of “one or more”, “at least one”, and “one or more than one”.

The use of the term “or” is used to mean “and/or” unless clearly indicated to refer solely to alternatives and only if the alternatives are mutually exclusive.

If the specification states a component or feature “may,” “can,” “could,” or “might” be included or have a characteristic, that particular component or feature is not required to be included or have the characteristic.

Throughout this disclosure, the term “about” is used to indicate that a value includes the inherent variation of error for the quantifying device, mechanism, or method, or the inherent variation that exists among the subject(s) to be measured. For example, but not by way of limitation, when the term “about” is used, the designated value to which it refers may vary by plus or minus ten percent, or nine percent, or eight percent, or seven percent, or six percent, or five percent, or four percent, or three percent, or two percent, or one percent, or one or more fractions therebetween.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The phrases “in one embodiment”, “in an embodiment”, “according to one embodiment”, and the like generally mean the particular feature, structure, or characteristic following the phrase is included in at least one embodiment of the present disclosure, and may be included in more than one embodiment of the present disclosure. Importantly, such phrases are non-limiting and do not necessarily refer to the same embodiment but, of course, can refer to one or more preceding and/or succeeding embodiments. For example, in the appended claims, any of the claimed embodiments can be used in any combination.

In the processes described herein, the steps can be carried out in any order without departing from the principles of the invention, except when a temporal or operational sequence is explicitly recited.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

Values expressed in a range format should be interpreted in a flexible manner to include not only the numerical values explicitly recited as the limits of the range, but to also include all of the individual numerical values or sub-ranges encompassed within that range as if each numerical value and sub-range is explicitly recited. For example, a range such as from 1 to 6, should be considered to have specifically disclosed sub-ranges, such as, from 1 to 3, from 2 to 4, from 3 to 6, etc., as well as individual numbers within that range, for example, 1, 2, 3, 4, 5, and 6. This applies regardless of the breadth of the range.

The terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the present disclosure.

The term “optional” or “optionally” means that the subsequently described event or circumstance may or may not occur, and that the description includes instances where said event or circumstance occurs and instances where it does not.

The present disclosure is generally directed to a process for preparing a cyclic urea derivative having a general formula

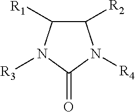

where R₁ and R₂ are independently selected from (a) hydrogen, (b) a straight-chain, branched or cyclic alkyl radical having 1 to 12 carbon atoms, which can be optionally substituted by C₆-C₁₀ aryl, F, Cl or Br, (c) a C₆-C₁₀ aryl radical, which can be optionally substituted by C₁-C₁₂ alkyl, (d) a heteroalkyl radical in which the alkyl radicals as defined in (b) are interrupted by one or more heteroatoms selected from O, S and N, and (e) a heteroaryl radical with 5 to 10 ring atoms which contain 1 to 3 heteroatoms selected from O, S and N and R₃ and R₄ are independently selected from (a) a straight-chain, branched or cyclic alkyl radical having 1 to 12 carbon atoms, which can be optionally substituted by C₆-C₁₀ aryl, F, Cl or Br, (b) a C₆-C₁₀ aryl radical, which can be optionally substituted by C₁-C₁₂ alkyl, (c) a heteroalkyl radical in which the alkyl radicals as defined in (a) are interrupted by one or more heteroatoms selected from O, S and N, and (d) a heteroaryl radical with 5 to 10 ring atoms which contain 1 to 3 heteroatoms selected from O, S and N comprising the step of reacting an alkylene carbonate with a primary amine in the presence of a metal oxide catalyst to form a reaction product comprising the cyclic urea derivative.

According to one embodiment, the alkylene carbonate is a compound having the formula (I):

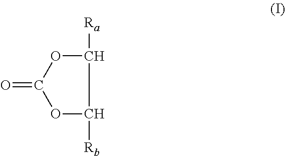

where Rₐ and R_{b} are independently hydrogen, hydroxymethyl, a straight or branched chain C₁-C₁₈ alkyl group, an aryl group or an alkylaryl group in which the benzene ring is substituted with a C₁-C₁₈ alkyl group.

In further embodiments, the alkylene carbonates for use in this disclosure are those of formula I above where Rₐ and R_{b} are independently hydrogen, methyl, ethyl, propyl or hydroxymethyl. Most preferred alkylene carbonates are those in which Rₐ and R_{b} are hydrogen (ethylene carbonate), methyl (propylene carbonate) and ethyl (butylene carbonate).

The following are examples of alkylene carbonates for use in this disclosure and including mixtures thereof: 1,3-dioxolan-2-one (also referred to as ethylene carbonate); 4-methyl-1,3-dioxolan-2-one (also referred to as propylene carbonate); 4-hydroxymethyl-1,3-dioxolan-2-one; 4,5-dimethyl-1,3-dioxolan-2-one; 4-ethyl-1,3-dioxolan-2-one; 4,4-dimethyl-1,3-dioxolan-2-one (previous three also referred to as butylene carbonates); 4-methyl-5-ethyl-1,3-dioxolan-2-one; 4,5-diethyl-1,3-dioxolan-2-one; 4,4-diethyl-1,3-dioxolan-2-one; 1,3-dioxan-2-one; 4,4-dimethyl-1,3-dioxan-2-one; 5,5-dimethyl-1,3-dioxan-2-one; 5,5-dihydroxymethyl-1,3-dioxan-2-one; 5-methyl-1,3-dioxan-2-one; 4-methyl-1,3-dioxan-2-one; 5-hydroxy-1,3-dioxan-2-one; 5-hydroxymethyl-5-methyl-1,3-dioxan-2-one; 5,5-diethyl-1,3-dioxan-2-one; and 5-methyl-5-propyl-1,3-dioxan-2-one.

In one embodiment, the primary amine which is reacted with the alkylene carbonate is a compound having the formula (II):

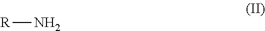

where R is (a) a straight-chain, branched or cyclic alkyl radical having 1 to 12 carbon atoms, which can be optionally substituted by C₆-C₁₀ aryl, F, Cl, Br; (b) a C₆-C₁₀ aryl radical, which can be optionally substituted by C₁-C₁₂ alkyl; (c) a heteroalkyl radical in which the alkyl radicals as defined in (a) are interrupted by one or more heteroatoms selected from O, S and N; and (d) a heteroaryl radical with 5 to 10 ring atoms which contain 1 to 3 heteroatoms selected from O, S and N.

Examples of primary amines include, but are not limited to, methylamine, ethylamine, propylamine, butylamine, heptylamine, hexylamine, cyclopropylamine, cyclohexylamine, cycloheptylamine, cyclooctylamine aniline, N-methylaniline, N,N-dimethylaniline, m-toluidine, o-chloroaniline, 3,5-dimethylaniline, o-anisidine, 2,5-dichloroaniline, 2,4,6-trichloroaniline, 3,4-dichloroaniline, benzylamine and mixtures thereof.

In one embodiment, the primary amine is preferably a monoalkylamine in which the alkyl group has 1 to 8 carbon atoms and more preferably 1 to 4 carbon atoms, especially methylamine.

The reaction between the alkylene carbonate and primary amine takes place in the presence of a metal oxide catalyst. In one embodiment, the metal oxide catalyst is an alkaline oxide or an alkaline earth oxide including, for example, lithium oxide, sodium oxide, potassium oxide, cesium oxide, magnesium oxide, calcium oxide, strontium oxide, barium oxide, rubidium oxide and mixtures thereof. In another embodiment, the metal oxide catalyst is an alkaline oxide catalyst comprising lithium oxide, sodium oxide, potassium oxide, cesium oxide and a combination thereof. In still another embodiment, the metal oxide catalyst is cesium oxide.

The reaction between the alkylene carbonate and primary amine may take place in the absence or presence of a solvent which does not participate in the reaction. In one embodiment, the solvent is water which may be present in an amount of up to about 50% by weight, based on the total weight of the alkylene carbonate. In another embodiment, the amount of water present is about 30% by weight to about 45% by weight, based on the total weight of alkylene carbonate.

In some embodiments, the molar ratio of the alkylene carbonate to the primary amine (alkylene carbonate:primary amine) present during the reaction may be up to about 1:5, such as from about 0.5:2.5, to about 1:2 or from about 0.8:1 to about 1:1.5, or from about 0.9:1 to about 1.1:1. In other embodiments, the molar ratio of the alkylene carbonate to the primary amine present during the reaction may be from about 0.95:1 to about 1.05:1, or from about 0.97:1 to about 1.03:1, or from about 0.99:1 to about 1.01:1. The order of addition of the alkylene carbonate and the primary amine as raw materials is not particularly limited, and the alkylene carbonate may be added to the primary amine, or the primary amine may be added to the alkylene carbonate or the primary amine and the alkylene carbonate may be added at once.

In one embodiment, the amount of the metal oxide catalyst used during the reaction may be less than about 5% by weight, or less than about 3% by weight or less than about 2% by weight, or less than about 1.5% by weight, or less than about 1% by weight, or less than about 0.5% by weight, based on the total weight of the alkylene carbonate In other embodiments, the amount of the metal oxide catalyst used during the reaction may be from about 0.1% by weight to about 5% by weight, or from about 0.5% by weight to about 2.5% by weight, or from about 0.8% by weight to about 2% by weight, based on the total weight of the alkylene carbonate.

In some embodiments, the reaction between the alkylene carbonate and primary amine may take place at a temperature of from about 180° C. to about 300° C., or from about 220° C. to about 260° C. or from about 245° C. to about 255° C. The reaction between the alkylene carbonate and the primary amine may take place under pressure, or at reduced pressure, but is preferably carried out at atmospheric pressure. The time for the reaction to reach completion may be from about 0.5 hours to about 24 hours, or from about 1 hour to about 10 hours or from about 2 hours to about 5 hours. In addition to the desired cyclic urea derivative, one or more by-products may be present in the obtained reaction product (for e.g., one or more by-products such as glycols, piperazines and ureas, unreacted primary amine, unreacted alkylene carbonate, and other impurities). The cyclic urea derivative can be isolated from at least a portion of the one or more by-products and purified by subjecting the reaction product to a separation technique, such as a concentration, crystallization, recrystallization, distillation, fractional distillation, or a chromatography technique.

The percent yield of the cyclic urea derivative (i.e., 100×([measured amount of cyclic urea derivative, such as by gas chromatography]/[maximum amount of cyclic urea derivative that can be produced from the given amounts of alkylene carbonate and primary amine])) obtained by the process of the present disclosure may be at least about 65%, or at least about 70%, or at least about 80%, or at least about 85%. Particular examples of cyclic urea derivatives which may be produced according to the process of the present disclosure include, but are not limited to, 1,3-dimethyl-2-imidazolidinone, 1,3,4-trimethyl-2-imidazolidinone and 1,3-dipropyl-2-imidazolidinone.

The cyclic urea derivatives obtained by the process of the present disclosure may be used in a variety of ways, such as in the preparation of a lithium ion battery, as a photochemical reaction solvent, as a solvent for photoelectrochemical display elements, in an electrolyte solvent, in an electrolyte solvent for batteries, as a solvent for electrolytic reactions, as a solvent for electrolytic polymerization, as a solvent for electroplating, as a solvent for electrolytic polishing, as a solvent for cleaning the surface of a semiconductor wafer and to remove photoresist layer(s), as an aprotic polar solvent in organic synthesis reactions, as a solvent for polymerization or extraction, as a low toxic high-boiling solvent, as an industrial cleaning agent, as a solvent for peeling or removing a paint or coating film, as a finishing agent for textiles, and as a solvent in the preparation of automotive part cleaning formulations or pharmaceutical formulations or agricultural chemical formulations.

Examples of the present process for preparing cyclic urea derivatives are provided below. However, the present disclosure is to be understood to not be limited in its application to the specific experiments, results, and laboratory procedures disclosed herein below. Rather, the Examples are simply provided as one of various embodiments and are meant to be exemplary and not exhaustive.

### EXAMPLES

### Example 1—Process for Preparing 1,3-Dimethyl-2-Imidazolidinone without Using a Catalyst

A 1 L autoclave reactor, equipped with an agitator, nitrogen line and feed line, was purged with nitrogen and 250 grams of ethylene carbonate, 100 grams of DI water and 250 grams of monomethylamine were charged into the reactor. The reactor was slowly heated to a temperature of about 250° C. for about 30 minutes and the temperature was then maintained at 250° C. for about 24 hours. The reactor was then cooled to room temperature, and the resulting reaction product was pack finished into a 32 oz bottle, nitrogen pad. The contents of the reaction product were determined (via gas chromatography) and included 1,3-dimethyl-2-imidazolidinone at a yield of about 74% and by-products including, but not limited to, dimethylpiperazine, trimethylethanediamine, tetramethylethanediamine and ethylene glycol.

### Example 2—Process for Preparing 1,3-Dimethyl-2-Imidazolidinone with a Metal Oxide Catalyst

A 1 L autoclave reactor, equipped with an agitator, nitrogen line and feed line, was purged with nitrogen and 250 grams of ethylene carbonate, 100 grams of DI water, 5 grams of cesium oxide and 250 grams of monomethylamine were charged into the reactor. The reactor was slowly heated to a temperature of about 250° C. for about 30 minutes and the temperature was then maintained at 250° C. for about 24 hours. The reactor was then cooled to room temperature, and the resulting reaction product was pack finished into a 32 oz bottle, nitrogen pad. The contents of the reaction product were then determined (via gas chromatography) and included 1,3-dimethyl-2-imidazolidinone at a yield of about 88% and by-products including, but not limited to, dimethylpiperazine. The amount of impurities was significantly reduced when a metal oxide catalyst was used as compared to Example 1.

### Example 3—Process for Preparing 1,3-Dimethyl-2-Imidazolidinone Using a Metal Oxide Catalyst

A 1 L autoclave reactor, equipped with an agitator, nitrogen line and feed line, was purged with nitrogen and 250 grams of ethylene carbonate, 100 grams of DI water, 5 grams of cesium oxide and 250 grams of monomethylamine were charged into the reactor. The reactor was slowly heated to a temperature of about 180° C. for about 30 minutes and the temperature was then maintained at 180° C. for about 24 hours. The reactor was then cooled to room temperature, and the resulting reaction product was pack finished into a 32 oz bottle, nitrogen pad. The contents of the reaction product were then determined (via gas chromatography) and included 1,3-dimethyl-2-imidazolidinone at a yield of about 24% and by-products including, but not limited to, ethylene glycol (about 41% yield) and dimethyl urea (about 34% yield).

### Example 4—Process for Preparing 1,3-Dimethyl-2-Imidazolidinone Using a Metal Oxide Catalyst

A 1 L autoclave reactor, equipped with an agitator, nitrogen line and feed line, was purged with nitrogen and 250 grams of ethylene carbonate, 100 grams of DI water, 5 grams of cesium oxide and 250 grams of monomethylamine were charged into the reactor. The reactor was slowly heated to a temperature of about 220° C. for about 30 minutes and the temperature was then maintained at 220° C. for about 24 hours. The reactor was then cooled to room temperature, and the resulting reaction product was pack finished into a 32 oz bottle, nitrogen pad. The contents of the reaction product were then determined (via gas chromatography) and included 1,3-dimethyl-2-imidazolidinone at a yield of about 70% and by-products including, but not limited to, ethylene glycol and dimethyl urea.

### Example 5—Process for Preparing 1,3,4-Trimethyl-2-Imidazolidinone without Catalyst

A 1 L autoclave reactor, equipped with an agitator, nitrogen line and feed line, was purged with nitrogen and 250 grams of propylene carbonate, 86 grams of DI water and 216 grams of monomethylamine were charged into the reactor. The reactor was slowly heated to a temperature of about 250° C. for about 30 minutes and the temperature was then maintained at 250° C. for about 24 hours. The reactor was then cooled to room temperature, and the resulting reaction product was pack finished into a 32 oz bottle, nitrogen pad. The contents of the reaction product were then determined (via gas chromatography) and included 1,3,4-trimethyl-2-imidazolidinone at a yield of about 10.5% and by-products including, but not limited to, propylene glycol (about 83% yield).

### Example 6—Process for Preparing 1,3,4-Trimethyl-2-Imidazolidinone Using a Metal Oxide Catalyst

A 1 L autoclave reactor, equipped with an agitator, nitrogen line and feed line, was purged with nitrogen and 250 grams of propylene carbonate, 86 grams of DI water, 5 grams of cesium oxide and 216 grams of monomethylamine were charged into the reactor. The reactor was slowly heated to a temperature of about 250° C. for about 30 minutes and the temperature was then maintained at 250° C. for about 24 hours. The reactor was then cooled to room temperature, and the resulting reaction product was pack finished into a 32 oz bottle, nitrogen pad. The contents of the reaction product were then determined (via gas chromatography) and included 1,3,4-trimethyl-2-imidazolidinone at a yield of about 48% and by-products including, but not limited to, propylene glycol (about 32%).

From the above description, it is clear that the present disclosure is well adapted to carry out the object and to attain the advantages mentioned herein as well as those inherent in the present disclosure. While exemplary embodiments of the present disclosure have been described for the purposes of the disclosure, it will be understood that numerous changes may be made which will readily suggest themselves to those skilled in the art which can be accomplished without departing from the scope of the present disclosure and the appended claims.

What is claimed is:

1. A process for preparing a cyclic urea derivative having a general formula 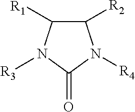
where R₁ and R₂ are independently selected from (a) hydrogen, (b) a straight-chain, branched or cyclic alkyl radical having 1 to 12 carbon atoms which can be optionally substituted by C₆-C₁₀ aryl, F, Cl or Br, (c) a C₆-C₁₀ aryl radical, which can be optionally substituted by C₁-C₁₂ alkyl, (d) a heteroalkyl radical in which the alkyl radicals as defined in (b) are interrupted by one or more heteroatoms selected from O, S and N, and (e) a heteroaryl radical with 5 to 10 ring atoms which contain 1 to 3 heteroatoms selected from O, S and N and R₃ and R₄ are independently selected from (a) a straight-chain, branched or cyclic alkyl radical having 1 to 12 carbon atoms which can be optionally substituted by C₆-C₁₀ aryl, F, Cl or Br, (b) a C₆-C₁₀ aryl radical, which can be optionally substituted by C₁-C₁₂ alkyl, (c) a heteroalkyl radical in which the alkyl radicals as defined in (a) are interrupted by one or more heteroatoms selected from O, S and N, and (d) a heteroaryl radical with 5 to 10 ring atoms which contain 1 to 3 heteroatoms selected from O, S and N comprising reacting an alkylene carbonate with a primary amine in the presence of a metal oxide catalyst to form a reaction product comprising the cyclic urea derivative.

2. The process according to claim 1, wherein the alkylene carbonate is a compound having the formula (I): 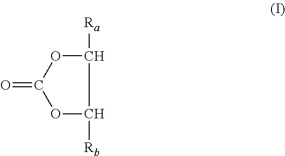
wherein Rₐ and R_{b} are independently hydrogen, hydroxymethyl, a straight or branched chain C₁-C₁₈ alkyl group, an aryl group or an alkylaryl group in which the benzene ring is substituted with a C₁-C₁₈ alkyl group.

3. The process according to claim 2, wherein Rₐ and R_{b} are independently hydrogen, methyl, ethyl or propyl.

4. The process according to claim 3, wherein Rₐ and R_{b} are hydrogen or methyl.

5. The process according to claim 1, wherein the primary amine is a compound having the formula (II): 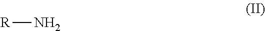
where R is (a) a straight-chain, branched or cyclic alkyl radical having 1 to 12 carbon atoms which can be optionally substituted by C₆-C₁₀ aryl, F, Cl, Br; (b) a C₆-C₁₀ aryl radical which can be optionally substituted by C₁-C₁₂ alkyl; (c) a heteroalkyl radical in which the alkyl radicals as defined in (a) are interrupted by one or more heteroatoms selected from O, S and N; and (d) a heteroaryl radical with 5 to 10 ring atoms which contain 1 to 3 heteroatoms selected from O, S and N.

6. The process according to claim 5, wherein the primary amine is methylamine, ethylamine, propylamine, butylamine, heptylamine, hexylamine, cyclopropylamine, cyclohexylamine, cycloheptylamine, cyclooctylamine aniline, N-methylaniline, N,N-dimethylaniline, m-toluidine, o-chloroaniline, 3,5-dimethylaniline, o-anisidine, 2,5-dichloroaniline, 2,4,6-trichloroaniline, 3,4-dichloroaniline, or benzylamine.

7. The process according to claim 6, wherein the primary amine comprises methylamine.

8. The process according to claim 1, wherein the metal oxide catalyst comprises an alkaline oxide or an alkaline earth oxide.

9. The process according to claim 8, wherein the metal oxide catalyst comprises lithium oxide, sodium oxide, potassium oxide, cesium oxide, magnesium oxide, calcium oxide, strontium oxide, barium oxide, rubidium oxide and mixtures thereof.

10. The process according to claim 9, wherein the metal oxide catalyst comprises potassium oxide, cesium oxide or rubidium oxide.

11. A process for preparing a cyclic urea derivative comprising reacting an alkylene carbonate with a primary amine in the presence of an alkaline metal oxide catalyst and a solvent to form a reaction product comprising the cyclic urea derivative and one or more by-products and optionally subjecting the reaction product to a separation technique to remove at least a portion of the one or more by-products.

12. The process according to claim 11, wherein the alkylene carbonate and the primary amine are present during the reaction at a molar ratio of alkylene carbonate:primary amine of from about 0.5:2.5 to about 1:2.

13. The process according to claim 11, wherein the amount of the alkaline metal oxide catalyst present during the reaction is from about 0.1% by weight to about 5% by weight, based on the total weight of the alkylene carbonate.

14. The process according to claim 11, wherein the solvent comprises water.

15. The process according to claim 14, the water present in an amount of up to about 50% by weight, based on the total weight of the alkylene carbonate.

16. The process according to claim 11, wherein the reaction takes place at a temperature from about 245° C. to about 255° C.

17. The process according to claim 11, wherein the separation technique is a concentration, crystallization, recrystallization, distillation, fractional distillation, or chromatography technique.

18. The process according to claim 11, wherein the cyclic urea derivative is 1,3-dimethyl-2-imidazolidinone.

19. The process according to claim 18, wherein the 1,3-dimethyl-2-imidazolidinone is present in the reaction product at a percent yield of at least 80%.
